(19) United States
(12) Patent Application Publication
(10) Pub. No.: US 2004/0034013 A1
(43) Pub. Date: Feb. 19, 2004

(54) Methods of treatment

(76) Inventors: Maxwell D Cummings, Strafford, PA (US); Robert W Marquis JR., Wayne, PA (US); Yu Ru, Wayne, PA (US); Scott K Thompson, Phoenixville, PA (US); Daniel F Veber, Ambler, PA (US); Dennis S Yamashita, Wayne, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/258,412

(22) PCT Filed: Apr. 17, 2001
(86) PCT No.: PCT/US01/12386

(51) Int. Cl. A61K 31/55

(52) U.S. Cl. 514/217.04; 514/217.11

(57) ABSTRACT

The present invention provides methods which use 4-amino-azepan-3-one protease inhibitors of cathepsin L in the treatment of diseases in which cathepsin L is implicated, especially treatment or prevention of rheumatoid arthritis; treatment or prevention of cancer metastasis; treatment or prevention of diseases requiring inhibition of tissue destruction by macrophage, particularly lung macrophage, such as asthma, chronic obstructive pulmonary disease (COPD), and emphysema; treatment or prevention of diseases requiring, for therapy, inhibition of positive selection of CD4+ T-cells by cortical thymic epithelial cells.

### FIELD OF THE INVENTION

This invention relates in general to the use of 4-amino-azepan-3-one protease inhibitors, particularly such inhibitors of cathepsin L, in the treatment of diseases in which cathepsin L is implicated, especially in the treatment or prevention of rheumatoid arthritis; treatment or prevention of cancer metastasis; treatment or prevention of diseases requiring inhibition of tissue destruction by macrophage, particularly lung macrophage, such as asthma, chronic obstructive pulmonary disease (COPD), and emphysema; treatment or prevention of diseases requiring, for therapy, inhibition of positive selection of CD4⁺T₋ cells by cortical thymic epithelial cells.

### BACKGROUND OF THE INVENTION

Cathepsins are a family of enzymes which are part of the papain superfamily of cysteine proteases. Cathepsins B, H, L, N and S have been described in the literature.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458.

Pathological levels of cathepsin L have been implicated in several disease states. Thus, selective inhibition of cathepsin L may provide an effective treatment for diseases requiring, for therapy or prevention: inhibition of rheumatoid arthritis (see Iwata et. al. Arthritis and Rheumatism 1997, 40, 499); inhibition of cancer metastasis (see K. Ishidoh and E. Kominami Biol. Chem. 1998, 379, 131; inhibition of tissue destruction by macrophage, particularly lung macrophage, in diseases such as asthma, chronic obstructive pulmonary disease (COPD), and emphysema (see Chapman H A Jr; Munger J S; Shi G P American Journal of Respiratory and Critical Care Medicine 1994, 150(6 Pt 2), S155-9); and inhibition of positive selection of CD4⁺T⁻ cells by cortical thymic epithelial cells (Nakagawa Science 1998, 270, 450).

Several cysteine protease inhibitors are known. Palmer, (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and and European Patent Application No. EP 0 623 592 A1 describe alkoxymethyl and mercaptomethyl ketones which inhibit the cysteine protease IL-1βconvertase. Alkoxymethyl and mercaptomethyl ketones have also been described as inhibitors of the serine protease kininogenase (International Patent Application No. PCT/GB91/01479).

Azapeptides which are designed to deliver the azaamino acid to the active site of serine proteases, and which possess a good leaving group, are disclosed by Elmore et al., Biochem. J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984, 259, 4288, and are known to inhibit serine proteases. In addition, J. Med. Chem., 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta,, 701, 328).

1,3-diamido-propanones have been described as analgesic agents in U.S. Pat. Nos. 4,749,792 and 4,638,010.

A variety of cysteine and serine protease inhibitors, especially of cathepsin K, have been disclosed in International Publication Number WO 97/16433 , published on May 9, 1997.

We have now discovered that certain 4-amino-azepan-3-one compounds inhibit cathepsin L, and are useful in the treatment of diseases in which cathepsin L is implicated.

### SUMMARY OF THE INVENTION

An object of the present invention is to provide methods of treatment which use 4-amino-azepan-3-one carbonyl protease inhibitors of cathepsin L of Formula I and which are useful for treating diseases which may be therapeutically modified by altering the activity of cathepsin L.

In a particular aspect, the methods of this invention are especially useful for treatment or prevention of rheumatoid arthritis; treatment or prevention of cancer metastasis; treatment or prevention of diseases requiring inhibition of tissue destruction by macrophage, particularly lung macrophage, such as asthma, chronic obstructive pulmonary disease (COPD), and emphysema; treatment or prevention of diseases requiring, for therapy, inhibition of positive selection of CD4⁺T⁻ cells by cortical thymic epithelial cells.

### DETAILED DESCRIPTION OF THE INVENTION

The present invention provides a method of inhibiting cathepsin L comprising administering to an animal, particularly a mammal, most particularly a human being in need thereof, an effective amount of a compound of Formula I:

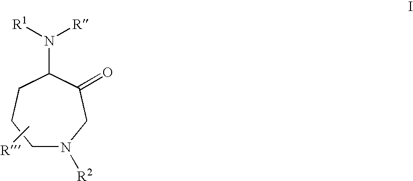

wherein:

R¹ is selected from the group consisting of:

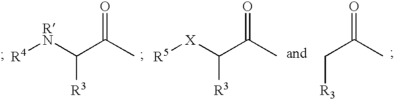

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—,

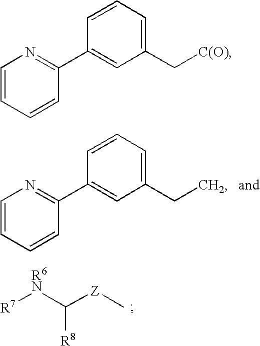

R⁹R¹¹NC(O)—, R⁹R¹¹NC(S)—, R⁹(R¹¹)NSO₂—

R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R³ and R′ may be connected to form a pyrrolidine, piperidine or morpholine ring;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;

R⁶ is selected from the group consisting of: H, C₁₋₆alkyl, C₃₋₆cycloalkyl-C₀₋₆alkyl, Ar—C₀₋₆alkyl, and Het-C₀₋₆alkyl;

R⁷ is selected from the group consisting of; H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, and R¹⁰R¹⁴NC(S)—;

R⁸ is selected from the group consisting of: H, C₁₋₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl and Het-C₀₋₆alkyl;

R¹⁰ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R¹⁴ is selected from the group consisting of: H, C ₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, C ₁₋₆alkyl, Ar—C₀₋₆alkyl, or Het-C₀₋₆alkyl;

R′″ is selected from the group consisting of: H, C₁₋₆alkyl, C₃₋₆cycloalkyl-C₀₋₆alkyl, Ar—C₀₋₆alkyl, and Het-C₀₋₆alkyl;

X is selected from the group consisting of: CH₂, S, and O;

Z is selected from the group consisting of: C(O) and CH₂;

and pharmaceutically acceptable salts, hydrates and solvates thereof.

In compounds of Formula I, R¹ is preferably

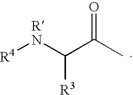

In such compounds:

R³ is selected from the group consisting of: H, C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl, preferably C₁₋₆alkyl and Ar—C₀₋₆alkyl, most preferably isobutyl, napthalen-2-ylmethyl, benzyl, and benzyloxymethyl;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—, preferably R⁵C(O)—.

R⁵ is selected from the group consisting of: C₁₋₆alkyl, C₂₋₆alkenyl, C₂₋₆alkynyl, C₃₋₆cycloalkyl-C₀₋₆alkyl, Ar—C₀₋₆alkyl or Het-C₀₋₆alkyl. Preferably R⁵ is selected from the group consisting of: C₁₋₆alkyl, Ar—C₀₋₆alkyl and Het-C₀₋₆alkyl. More preferably R⁵ is selected from the group consisting of:

quinolinyl, especially quinolin-2-yl, quinolin-4-yl and quinolin-8-yl;

isoquinolinyl, especially isoquinolin-1-yl;

naphthalenyl, especially naphthalen-1-yl; and

benzofuranyl, especially benzofuran-2-yl.

R′ is selected from the group consisting of: H, C₁₋₆alkyl, Ar—C₀₋₆alkyl, and Het-C₀₋₆alkyl, preferably H.

R″ selected from the group consisting of: H, C₁₋₆alkyl, Ar—C₀₋₆alkyl, and Het-C₀₋₆alkyl, preferably H.

In compounds of Formula I, R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—,

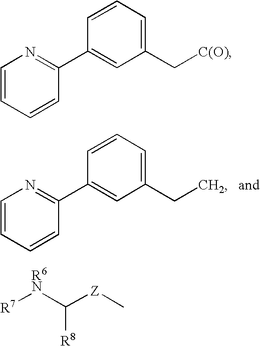

Preferably R² is R⁹SO₂.

R⁹ is selected from the group consisting of: C₁₋₆alkyl, C₃₋₆cycloalkyl-C₀₋₆alkyl, Ar—C₀₋₆alkyl, and Het-C₀₋₆alkyl, preferably Het-C₀₋₆alkyl, more preferably pyridinyl and 1-oxy-pyridinyl. When R² is R⁹SO₂, R⁹ is even more preferably selected from the group consisting of: pyridin-2-yl and 1-oxy-pyridin-2-yl. Most preferably, R⁹ is 1-oxy-pyridin-2-yl.

Most preferred are compounds of Formula I wherein:

R¹ is

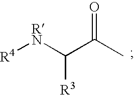

R² is R⁹SO₂;

R³ is selected from the group consisting of: isobutyl, napthalen-2-ylmethyl, benzyl, and benzyloxymethyl;

R⁴ is R⁵C(O);

R⁵ is selected from the group consisting of: quinolin-2-yl, quinolin-4-yl, quinolin-8-yl, isoquinolin-1-yl, naphthalen-1-yl, and benzofuran-2-yl;

R⁹ is selected from the group consisting of: pyridin-2-yl and 1-oxy-pyridin-2-yl, preferably 1-oxy-pyridin-2-yl.

R′ is H

R″ is H; and

R′″ is H;

Compounds of Formula I selected from the following group are particularly preferred embodiments for use in the present invention:

Quinoline-8-carboxylic acid {(S)-3-methyl-1-[3-oxo-1-(pyridine-2-sulfonyl)-azepan-4-ylcarbamoyl]-butyl}amide;

Quinoline-4-carboxylic acid {(S)-3-methyl-1-[3-oxo-1-(pyridine-2-sulfonyl)-azepan-4-ylcarbamoyl]-butyl}amide;

Isoquinoline-1-carboxylic acid {(S)-3-methyl-1-[3-oxo-1-(pyridine-2-sulfonyl)-azepan-4-ylcarbamoyl]-butyl}amide;

Quinoline-8-carboxylic acid {(S)-2-naphthylen-2-yl-1-[3-oxo-1-(pyridine-2-sulfonyl)-azepan-4-ylcarbamoyl)-ethyl]-amide;

Naphthylene-1-carboxylic acid {(S)-2-naphthylen-2-yl-1-[3-oxo-1-(pyridine-2-sulfonyl)-azepan-4-ylcarbamoyl)-ethyl]-amide;

Quinoline-8-carboxylic acid {(S)-1-[3-oxo-1-(pyridine-2-sulfonyl)-azepan-4-ylcarbamoyl]-2-phenyl-ethyl}-amide;

Naphthylene-1-carboxylic acid {(S)-1-[3-oxo-1-(pyridine-2-sulfonyl)-azepan-4-ylcarbamoyl]-2-phenyl-ethyl}-amide;

Quinoline-2-carboxylic acid {(S)-1-[3-oxo-1-(pyridine-2-sulfonyl)-azepan-4-ylcarbamoyl]-2-phenyl-ethyl}-amide;

Benzofuran-2-carboxylic acid {(S)-1-[3-oxo-1-(pyridine-2-sulfonyl)-azepan-4-ylcarbamoyl]-2-phenyl-ethyl}-amide;

Benzofuran-2-carboxylic acid {(S)-2-naphthylen-2-yl-1-[3-oxo-1-(pyridine-2-sulfonyl)-azepan-4-ylcarbamoyl)-ethyl]-amide; and

Benzofuran-2-carboxylic acid {(S)-2-benzyloxy-1-[3-oxo-1-(pyridine-2-sulfonyl)-azepane-4-ylcarbamoyl]-ethyl }-amide.

Specific representative compounds used in the present invention are set forth in Examples 1-12.

Compared to the corresponding 5 and 6 membered ring compounds, the 7 membered ring compounds used in the present invention are configurationally more stable at the carbon center alpha to the ketone.

The present invention also uses deuterated analogs of the inventive compounds. A representative example of such a deuterated compound is set forth in Example 12. A representative synthetic route for the deuterated compounds of the present invention is set forth in Example 12, below. The deuterated compounds used in the present invention exhibit superior chiral stability compared to the protonated isomer.

### Definitions

The compounds used in the present invention include all hydrates, solvates, complexes and prodrugs. Prodrugs are any covalently bonded compounds which release the active parent drug according to Formula I in vivo. If a chiral center or another form of an isomeric center is present in a compound used in the present invention, all forms of such isomer or isomers, including enantiomers and diastereomers, are intended to be covered herein. Compounds used in the present methods containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR¹², C(O)R¹², SR¹², S(O)R¹², NR¹²₂, R¹²NC(O)OR⁵, CO₂R¹², CO₂NR¹²₂, N(C═NH)NH₂, Het, C₃₋₆cycloalkyl, and Ar; where R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl; and R¹² is selected from the group consisting of: H, C₁₋₆alkyl, Ar—C₀₋₆alkyl, and Het-C₀₋₆alkyl;

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR¹⁵R¹⁶; O(CH₂)₁₋₆NR¹⁵R¹⁶; C ₁₋₆alkyl, OR¹⁷, N(R¹⁷)₂, SR¹⁷, CF₃, NO₂, CN, CO₂R¹⁷, CON(R¹⁷), F, Cl, Br or I; where R¹⁵ and R¹⁶ are H, C₁₋₆alkyl, Ph-C₀₋₆alkyl, naphthyl-C₀₋₆alkyl or Het-C₀₋₆alkyl; and R¹⁷ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR¹⁷, N(R¹⁷)₂, SR¹⁷, CF₃, NO₂, CN, CO₂R¹⁷, CON(R¹⁷), F, Cl, Br and I, where R¹⁷ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8 naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

### Methods of Preparation

Compounds of the general formula I may be prepared in a fashion analogous to that outlined in Schemes 1, 2 and 3. Alkylation of benzyl-N-allylcarbamate (1) with a base such as sodium hydride and 5-bromo-1-pentene provides the diene 2 (Scheme 1). Treatment of 2 with bis(tricyclohexylphosphine)benzylidine ruthenium (IV) dichloride catalyst developed by Grubbs provides the tetrahydroazepine 3. Epoxidation of 3 may be effected with an oxidizing agent common to the art such as m-CPBA to provide the epoxide 4. Nucleophilic ring opening of epoxide 4 may be effected with a reagent such as sodium azide to provide the azido alcohol 5 which may be reduced to the amino alcohol 6 under conditions common to the art such as 1,3-propanedithiol and triethylamine in methanol or triphenylphosphine in THF and water. The amine of compound 6 may be protected with with di-tert-butyl dicarbonate to provide derivative 7 (Scheme 2). Removal of the benzyloxycarbonyl protecting group may be effected by treatment of 7 with hydrogen gas in the presence of a catalyst such as 10% Pd/C to provide the amine 8. Treatment of amine 8 with a sulfonyl chloride such as 2-pyridinesulfonyl chloride in the presence of a base such as triethylamine provides the sulfonamide derivative 9. Removal of the tert-butoxycarbonyl protecting group may be effected with an acid such as hydrochloric acid to provide intermediate 10. Coupling of 10 with an acid such as N-Boc-phenylalanine in the presence of a coupling agent common to the art such as HBTU or polymer supported EDC provides the alcohol intermediate 11. Removal of the tert-butoxycarbonyl protecting group under acidic conditions provides 12. Coupling of 12 with an acid such as benzofuran-2-carboxylic acid in the presence of a coupling agent such as HBTU or polymer supported EDC provides alcohol 13. Alcohol 13 may be oxidized with an oxidant common to the art such as pyridine sulfur trioxide complex in DMSO and triethylamine or the Dess-Martin periodinane to provide the ketone 14. The diastereomers of 14 may be separated by HPLC.

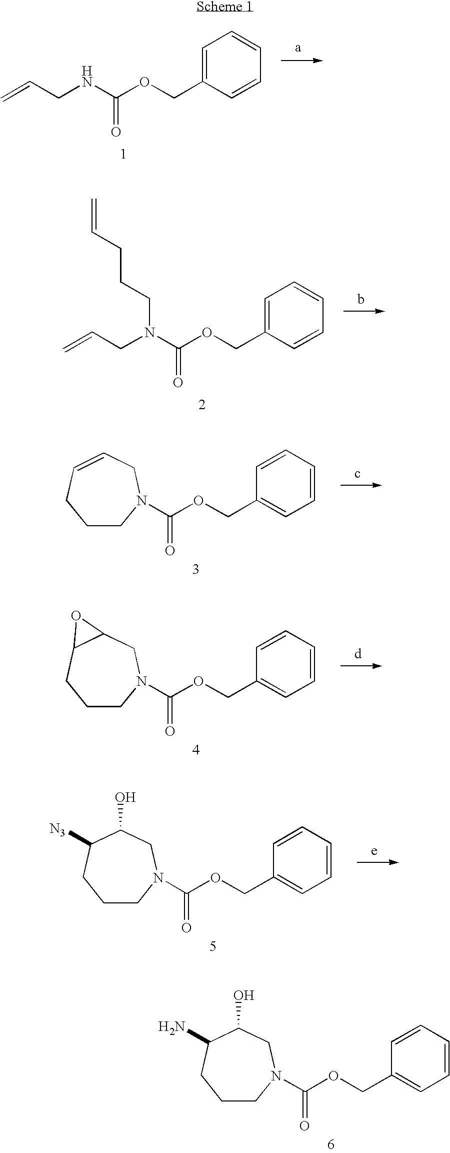

Reagents and Conditions:

(a) NaH, 5-bromo-1-pentene, NaH; (b) bis(tricyclohexylphosphine)benzylidine ruthenium (IV) dichloride, CH₂Cl₂, reflux; (c) mn-CPBA, CH₂Cl₂; (d) NaN₃, NH₄Cl, CH₃OH, H₂O; (e) TEA, 1,3-propanedithiol, CH₃OH.

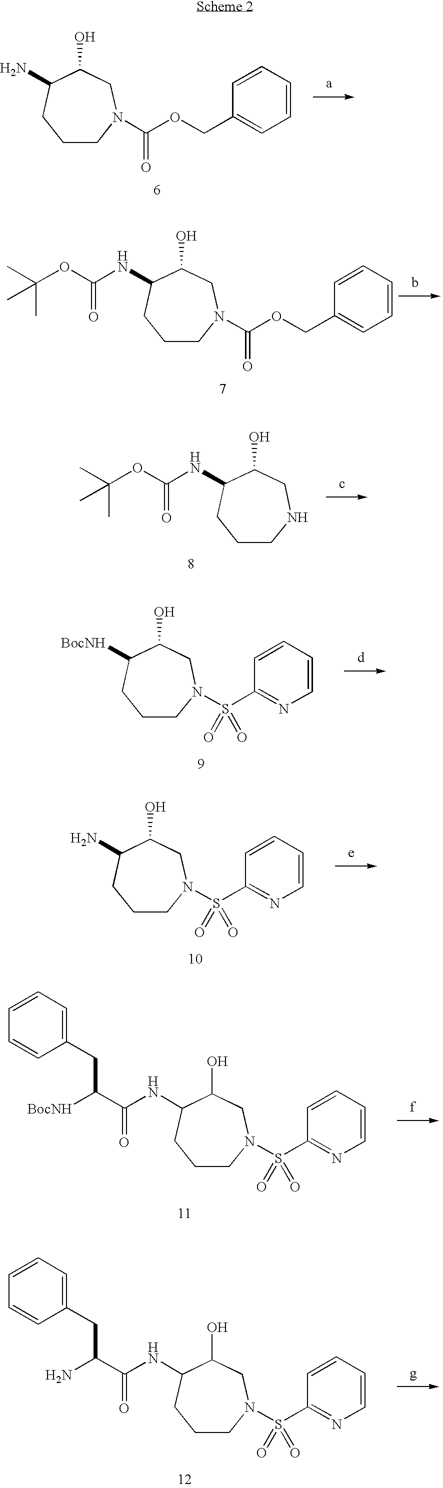

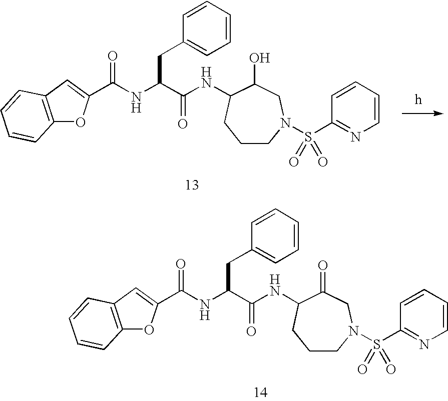

Reagents and Conditions:

(a) Di-tert-butyl dicarbonate, THF; (b) H₂, 10% Pd/C, EtOAc; (c) 2-pyridinesulfonyl chloride, TEA, CH₂Cl₂; (d) HCl, EtOAc; (e) N-Boc-phenylalanine, P-EDC, CH₂Cl₂; (f) HCl, CH₂Cl₂; (g) benzofuran-2-carboxylic acid, P-EDC, CH₂Cl₂; (h) Dess-Martin periodinane, methylene chloride.

Alternatively compounds for the general formula I may be prepared as shown in Scheme 3. Acylation of the amino alcohol 6 with an acid such as N-Boc-leucine in the presence of a coupling agent such as EDC or HBTU provides the amide 15. Hydrogenolysis of the carbonylbenzyloxy protecting group employing conditions known in the art such as hydrogen gas in the presence of a catalyst such as 10% Pd/C gives the amine 16. Treatment of amine 16 with a sulfonyl chloride such as 2-pyridinesulfonyl chloride in the presence of a base

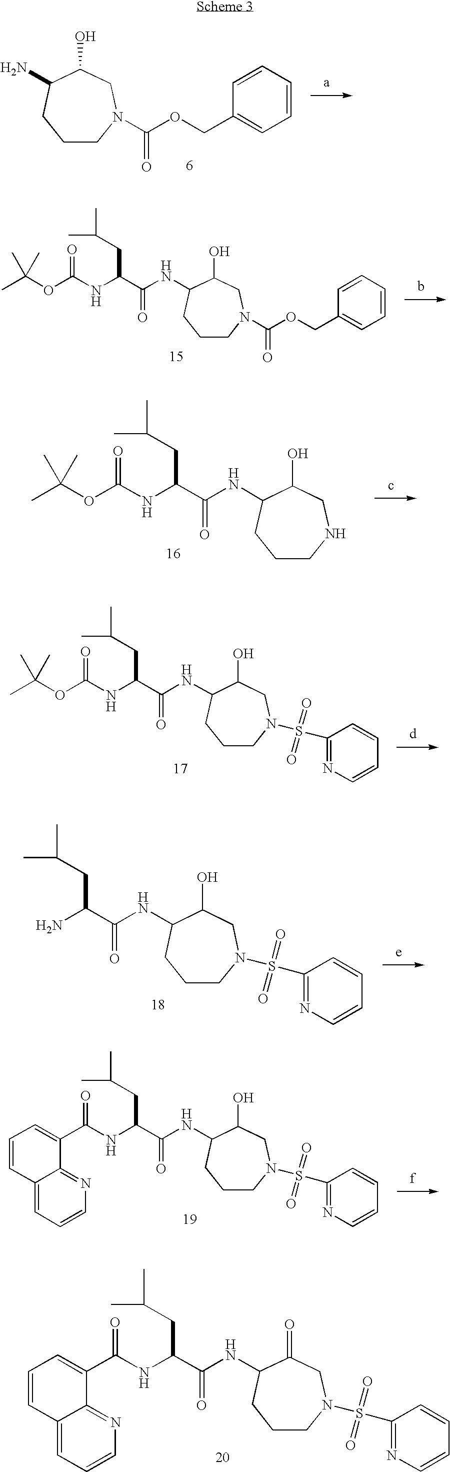

Reagents and Conditions:

(a) N-Boc-leucine, EDC, HOBt, TEA, CH₂Cl₂; (b) H₂, 10% Pd/C, EtOAc; (c) 2-pyridinesulfonyl chloride, TEA, CH₂Cl₂; (d) HCl, methanol; (e) quinoline-8-carboxylic acid, EDC, HOBt, TEA, CH₂Cl₂; (f) pyridine sulfur tri oxide complex, TEA, DMSO.

such as triethylamine provides the sulfonamide derivative 17. Removal of the tert-butoxycarbonyl protecting group may be effected with an acid such as hydrochloric acid to provide intermediate 18. Coupling of 18 with an acid such as quinoline-8-carboxylic acid in the presence of a coupling agent common to the art such as HBTU or EDC provides intermediate 19. Alcohol 19 may be oxidized with an oxidant common to the art such as pyridine sulfur trioxide complex in DMSO and triethylamine or the Dess-Martin periodinane to provide the ketone 20. The diastereomers of 20 may be separated by HPLC.

The deuterated compound of the Example 12 may be conveniently prepared according to Scheme 4. The skilled artisan will understand from Example 12 and Scheme 4 how to make any of the the deuterated compounds of the present invention.

The individual diastereomers of benzofuran-2-carboxylic acid {(S)-3-methyl-1-[(2,2′,4-trideuterio)-3-oxo-1-(pyridine-2-sulfonyl)-azepan-4-ylcarbamoyl]-butyl }amide 22 and 23 may be prepared as outlined in Scheme 4.

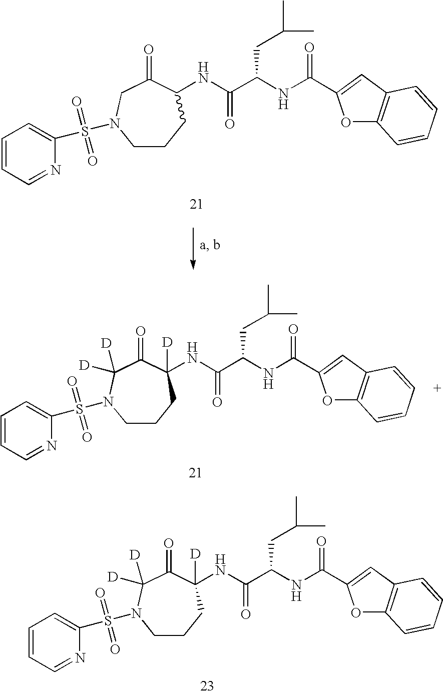

Reagents and Conditions:

a.) CD₃OD;D₂O (10:1), TEA; b.) HPLC separation.

Treatment of ketone 21 with triethylamine in CD₃OD:D₂O at reflux provides the deuterated analog as a mixture of diastereomers which are separated by HPLC to provide the deuterated compounds 22 and 23.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984. are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

The methods of the present invention may be practiced by administering a pharmaceutical composition which comprises a compound according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as inhibitors of cathepsin L. The present invention provides methods of treatment of diseases caused by pathological levels of cathepsin L, which methods comprise administering to an animal, particularly a mammal, most particularly a human in need thereof a therapeutically effective amount of an inhibitor of cathepsin L, including a compound of the present invention.

The present invention particularly provides methods for treating the following diseases in which cathepsin L is implicated:

diseases which require for therapy: inhibition of rheumatoid arthritis; inhibition of cancer metastasis; inhibition of tissue destruction by macrophage, particularly, lung macrophage, in diseases such as asthma, chronic obstructive pulmonary disease (COPD), and emphysema; and inhibition of positive selection of CD4⁺T⁻ cells by cortical thymic epithelial cells.

The present methods contemplate the use of one or more compounds of Formula I, alone or in combination with other therapeutic agents.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin S. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of Formula I may also be administered orally to the patient, in a manner such that the concentration of drug is sufficient to inhibit rheumatoid arthritis or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of Formula I are administered in accordance with the present methods.

### Biological Assays

The compounds used in the present methods may be tested in one of several biological assays to determine the concentration of compound which is required to have a given pharmacological effect.

Determination of Cathepsin L Proteolytic Catalytic Activity

All assays for cathepsin L were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemistry, 1989, 28, 140):

v=VₘA/[Kₐ(1+I/K_{i, app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ) [1−exp (−k_{obs}t)]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic examples, temperature is in degrees Centigrade (°C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Preparation of Quinoline-8-carboxylic Acid {(S)-3-methyl-1-{3-oxo-1-(pyridine-2-sulfonyl)-azepan-4-ylcarbamoyl]-butyl}Amide

a.) Allyl-pent-4-enyl-carbamic Acid Benzyl Ester

To a suspension of NaH (1.83 g, 76.33 mmol of 90% NaH) in DMF was added allyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2×'s), brine, dried (MgSO₄), filtered and concentrated. Column chromatography of the residue (10% ethyl acetate:hexanes) provided 10.3 grams of the title compound as an oil: MS(EI) 260 (M+H⁺).

b.) 2,3,4,7-Tetrahydro-azepine-1-carboxylic Acid Benzyl Ester

To a solution of compound of Example 1a (50 g) in dichloromethane was added bis(tricyclohexylphosphine)benzylidine ruthenium (IV) dichloride (5.0 g). The reaction was heated to reflux until complete as determined by TLC analysis. The reaction was concentrated in vacuo. Column chromatography of the residue (50% dichloromethane:hexanes) gave 35 g of the title compound: MS(EI) 232 (M+H⁺).

c.) 8-Oxa-3-aza-bicyclo[5.1.0]octane-3-carboxylic Acid Benzyl Ester

To a solution of the compound of Example 1b (35 g, 1.5 mol) in dichloromethane was added m-CPBA (78 g, 0.45 mol). The mixture was stirred overnight at room temperature whereupon it was filtered to remove the solids. The filtrate was washed with saturated water and saturated NaHCO₃ (several times). The organic layer was dried (MgSO₄), filtered and concentrated to give 35 g of the title compound which was of sufficient purity to carry on to the next step: MS(EI) 248 (M+H⁺), 270 (M+Na⁺).

d. 4-Azido-3-hydroxy-azepane-1-carboxylic Acid Benzyl Ester

To a solution of the epoxide from Example 1c (2.0 g, 8.1 mmol) in methanol:water (8:1 solution) was added NH₄Cl (1.29 g, 24.3 mmol) and sodium azide (1.58 g, 24.30 mmol). The reaction was heated to 65-75° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound: MS(EI) 291 (M+H⁺) plus 0.14 g of trans-4-hydroxy-3-azido-hexahydro-1H-azepine

e.) 4-Amino-3-hydroxy-azepane-1-carboxylic Acid Benzyl Ester

To a solution of the azido alcohol of Example 1d (1.1 g, 3.79 mmol) in methanol was added triethyamine (1.5 mL, 11.37 mmol) and 1,3-propanedithiol (1.1 mL, 11.37 mmoL). The reaction was stirred until complete consumption of the starting material was observed by TLC analysis whereupon the reaction was concentrated in vacuo. Column chromatography of the residue (20% methanol:dichloromethane) provided 0.72 g of the title compound: MS(EI) 265 (M+H⁺).

f.) 4-((S)-2-tert-Butoxycarbonylamino-4-methyl-pentanoylamino)-3-hydroxy-azepan-1-carboxylic Acid Benzyl Ester

To a solution of the amino alcohol of Example 1e (720 mg, 2.72 mmol) in CH₂Cl₂ was added EDC (521 mg), HOBt (368 mg) and N-Boc-leucine (630 mg). The reaction was maintained at room temperature until complete consumption of the starting material was observed by TLC analysis. The reaction was diluted with ethyl acetate and washed with 1N HCl, saturated K₂CO₃, water, brine, dried (MgSO₄), filtered and concentrated. Column chromatography of the residue (3% methanol:dichloromethane) gave 1.0 g of the title compound: MS(EI) 478 (M+H⁺).

g.) [(S)-1-(3-Hydroxy-azepan-4-ylcarbamoyl)-3-methyl-butyl]-carbamic Acid Tert Butyl Ester

To a solution of the compound of Example 1f (1.0 g) and 10% Pd/C (catalytic) in ethyl acetate:methanol (2:1 solution) was affixed a balloon of hydrogen. The reaction was stirred until complete consumption of the starting material was observed by TLC analysis. The reaction was filtered to remove the catalyst and the filtrate was concentrated to provide 0.82 g of the title compound: MS(EI) 344 (M+H⁺).

h.) {(S)-1-[3-Hydroxy-1-(pyridine-2-sulfonyl)-azepan-4ylcarbamoyl]-3-methyl-butyl }-carbamic Acid Tert-butyl Ester

Generation of 2-pyridinesulfonylchloride: A solution of 2-mercaptopyridine (2.23 g in 33 ml 9N HCl) was cooled to 0° C. Chlorine gas was bubbled into the solution for 90 min, taking care to maintain the temperature at 0° C. Ice cooled ethyl acetate was added followed by slow addition of ice cooled sat'd NaHCO₃ until the pH of the water layer was approximately 9. The organic layer were then washed with brine and dried over MgSO₄. Evaporation of the ethyl acetate gave 3.5 g of the crude 2-pyridinesulfonylchloride as a light yellow liquid.

To a solution of [(S)-1-(3-hydroxy-azepan-4-ylcarbamoyl)-3-methyl-butyl]-carbamic acid tert butyl ester of Example 1g (12 g, 34.93 mmol) in dichloromethane was added triethylamine (5.8 mL, 41.92 mmol) followed by the dropwise addition of 2-pyridinesulfonylchloride (7.45 g, 41.92 mmol). The reaction was stirred until complete as determined by TLC analysis. The mixture was then washed with sat. NaHCO₃, water, brine, dried (Na₂SO₄), filtered and concentrated. Column chromatography (75% ethyl acetate:hexanes to 100% ethyl acetate) of the residue provided 15 g of the title compound: MS 484 (M⁺)

i.) (S)-2-Amino-4-methyl-pentanoic Acid-[3-hydroxy-1-(pyridine-2-sulfonyl)-azepan-4-yl]-amide

To a solution of {(S)-1-[3-hydroxy-1-(pyridine-2-sulfonyl)-azepan-4-ylcarbamoyl]-3-methyl-butyl}-carbamic acid tert-butyl ester of Example 1h (14.3 g) in methanol was added 4 M HCl in dioxane. The reaction was stirred at room temperature until complete as determined by TLC analysis whereupon it was concentrated to provide 14 g of the title compound: MS (EI) 385 (M+H⁺).

j.) Quinoline-8-carboxylic Acid {(S)-3-methyl-1-[3-hydroxy-1-(pyridine-2-sulfonyl)-azepan-4-ylcarbamoyl]-butyl }Amide

To a solution of the compound of Example 1i (0.15 g, 0.33) in CH₂Cl₂ was added triethylamine (0.11 mL, 0.82 mmol), EDC (69 mg, 0.36 mmol), HOBt (49 mg, 0.36 mmol) and quinoline-8-carboxylic acid (62 mg, 0.36 mmol). The reaction was stirred until complete by TLC analysis. Workup and column chromatography of the residue gave 0.066 g of the title compound: MS(EI) 540 (M+H⁺).

k.) Quinoline-8-carboxylic Acid {(S)-3-methyl-1-[3-oxo-1-(pyridine-2-sulfonyl)-azepan-4-ylcarbamoyl]-butyl }Amide

To a solution of the alcohol of Example 1j (0.066 g, 0.12 mmol) in DMSO was added TEA (0.1 mL) and pyridine sulfur trioxide complex (57 mg). The reaction was stirred at room temperature for approximately 2 hours whereupon it was partitioned between ethyl acetate and water. The organic layer was washed with brine, dried, filtered and concentrated. Column chromatography of the residue (5% CH₃OH:CH₂Cl₂) provided the title compound as a mixture of diastereomers: ¹H NMR (CDCl₃): δ 1.0 (m, 6H), 1.5-2.1 (m, 5H), 2.2 (m, 2H), 2.7 (m, 1H), 3.7 (d, 1H), 4.0 (m, 1H), 4.7 (m, 2H), 5.0 (m, 1H), 7.5 (m, 4H), 7.6 (m, 1H), 7.7 (m, 3H), 8.2 (m, 1H), 8.6 (m, 1H), 8.7 (m, 1H), 8.9 (m, 1H); MS(EI): 538 (M+H⁺, 100%).

### Example 2

### Preparation of Quinoline-4-carboxylic Acid {(S)-3-methyl-1-[3-oxo-1-(pyridine-2-sulfonyl)-azepan-4-ylcarbamoyl]-butyl}amide

Following the procedures of Example 1j-k except substituting quinoline-4-carboxylic acid for quinoline-8-carboxylic acid of Example 1j the title compound was prepared: ¹H NMR (CDCl₃): δ 1.0 (m, 6H), 1.5-2.1 (m, 5H), 2.2 (m, 2H), 2.7 (m, 1H), 3.7 (d, 1H). 4.0 (m, 1H), 4.7 (m, 2H), 5.0 (m, 1H), 6.5-7.2 (m, 2H), 7.4 (m, 2H), 7.5 (m, 1H), 7.7 (m, 1H), 7.9 (m, 2H), 8.0 (m, 1H), 8.2 (m, 1H), 8.7 (m, 1H), 8.9 (m, 1H); MS(EI): 538 (M+H^{+,}100%)

The diastereomeric mixture was separated by HPLC to provide the faster eluting diastereoemer; MS(EI): 538 (M+H⁺, 100%), and the slower eluting diastereomer; MS(EI): 538 (M+H^{+,}100%).

### Example 3

### Preparation of Isoquinoline-1-carboxylic Acid {(S)-3-methyl-1-[3-oxo-1-(pyridine-2-sulfonyl)-azepan-4-ylcarbamoyl]-butyl}Amide

Following the procedures of Example 1j-k except substituting isoquinoline-1-carboxylic acid for quinoline-8-carboxylic acid of Example 1j the title compound was prepared: ¹H NMR (CDCl₃): δ 1.0 (m, 6H), 1.5-2.1 (m, 5H), 2.2 (m, 2H), 2.7 (m, 1H), 3.7 (d, 1H). 4.0 (m, 1H), 4.7 (m, 2H), 5.0 (m, 1H), 7.3 (m, 1H), 7.5 (m, 1H), 7.7-8.0 (m, 6H), 8.7 (m, 3H), 9.5 (m, 1H); MS(EI): 538 (M+H^{+,}100%).

The diastereomeric mixture was separated by HPLC to provide the faster eluting diastereoemer; MS(EI): 537 (M⁺, 100%), and the slower eluting diastereomer; MS(EI): 537 (M⁺, 100%).

### Example 4

### Preparation of Quinoline-8-carboxylic Acid {(S)-2-naphthylen-2-yl-1-[3-oxo-1-(pyridine-2-sulfonyl)-azepan-4-ylcarbamoyl)-ethyl]-amide

a.) 4-tert-Butoxycarbonylamino-3-hydroxy-azepane-1-carboxylic Acid Benzyl Ester

To a stirring solution of 4-amino-3-hydroxy-azepane-1-carboxylic acid benzyl ester (Example 1e, 1.04 g, 3.92 mmol) in THF was added di-tert-butyl dicarbonate (0.864 g). After stirring at room temperature for 30 minutes, the reaction mixture was diluted with diethyl ether and extracted with saturated NaHCO₃. The organic layer was dried over anhydrous Na₂SO₄, filtered, concentrated, and purified by silica gel column to give the title compound as a yellow oil (0.963 g, 2.64 mmol, 67%). MS (ESI): 365.03 (M+H⁺).

b.) 3-Hydroxy-azepan-4-yl-carbamic Acid-tert-butyl Ester

To a solution of 4-tert-butoxycarbonylamino-3-hydroxy-azepane-1-carboxylic acid benzyl ester (Example 4a, 0.963 g, 2.64 mmol) in ethyl acetate (16 mL) was added 10% palladium on carbon (500 mg). After stirring the solution at room temperature for 48 hours, the mixture was filtered through celite. The filterate was concentrated to yield the title compound (0.529 g, 2.29 mmol, 87%). MS(ESI): 231.92 (M+H⁺).

c.) 3-Hydroxy-1-(pyridine-2-sulfonyl)-azepan-4-yl-carbamic Acid-tert-butyl Ester

To a solution of 3-hydroxy-azepan-4-yl-carbamic acid-tert-butyl ester (Example 4b, 0.529, 2.29 mmol) in dichloromethane (20 mL) was added triethylamine (232 mg) and pyridine-2-sulfonyl chloride (410 mg, 2.32 mmol). After stirring at room temperature for 30 minutes, the mixture was washed with saturated NaHCO₃. The organic layer was dried, filtered, concentrated and purified on a silica gel column to give the title compound as a solid (0.583 g, 1.57 mmol, 68%); MS(ESI): 372.95 (M+H⁺).

d.) 4-Amino-1-(pyridine-2-sulfonyl)-azepan-3-ol

To a stirring solution of 3-hydroxy-1-(pyridine-2-sulfonyl)-azepan-4-yl-carbamic acid-tert-butyl ester (Example 4c, 0.583 g, 1.57 mmol) in ethyl acetate (0.5 mL) was added HCl (4M in dioxane) (3.9 mL). After stirring the reaction mixture for 30 minutes at room temperature, the mixture was concentrated to yield a white solid. The solid was treated with NaOH and then extracted with ethyl acetate. The organic layer was dried, filtered, and concentrated to yield a yellow solid (0.347 g, 1.28 mmol, 81%); MS (ESI) 272.93 (M+H⁺).

e.) {(S)-1-[3-hydroxy-1-(pyridine-2-sulfonyl)-azepan-4-ylcarbamoyl]-2-napthylene-2-yl-ethyl}-carbamic Acid Tert-butyl Ester

To a solution of the compound of Example 4d (225 mg) in dichloromethane was added TEA (0.15 mL), HOBt (99 mg), EDC (140 mg) and N-(t-butoxycarbonyl)-3-(2-naphthyl)-L-alanine (230 mg). The reaction was stirred until complete. Workup and column chromatography of the residue (3% methanol:dichloromethane) provided 0.35 g of the title compound: MS(ESI) 569 (M+H⁺).

f.) (S)-2-Amino-N-[3-hydroxy-1-(pyridine-2-sulfonyl)-azepan-4-yl]-3-naphthylen-2-yl-proprionamide

To a solution of the compound of Example 4e (0.35 g) in methanol (5 mL) was added HCl (5 mL of 4M HCl in dioxane). The reaction was stirred until complete by TLC analysis whereupon it was concentrated to provide 0.31 g of the title compound as a white solid.

g.) Quinoline-8-carboxylic Acid {(S)-2-naphthylen-2-yl-1-[3-hydroxy-1-(pyridine-2-sulfonyl)-azepan-4-ylcarbamoyl)-ethyl]-amide

To a solution of the compound of Example 4f (131 mg) in dichloromethane was added TEA, HOBt (39 mg), EDC (55 mg) and quinoline-8-carboxylic acid (51 mg). The reaction was stirred until complete. Workup and column chromatography of the residue (5% methanol:dichloromethane) provided the title compound: MS(ESI) 574 (M+H⁺).

h.) Quinoline-8-carboxylic Acid {(S)-2-naphthylen-2-yl-1-[3-oxo-1-(pyridine-2-sulfonyl)-azepan-4-ylcarbamoyl)-ethyl]-amide

Following the procedure of Example 1k except substituting the compound of Example 4g the title compound was prepared: MS 622 (M+H⁺).

### Example 5

### Preparation of Naphthylene-1-carboxylic Acid {(S)-2-naphthylen-2-yl-1-[3-oxo-1-(pyridine-2-sulfonyl)-azepan-4-ylcarbamoyl)-ethyl]-amide

Following the procedures of Example 4g-h except substituting naphthylene-1-carboxylic acid for quinoline-8-carboxylic acid the title compound was prepared: MS 621 (M+H⁺).

### Example 6

### Preparation of Naphthylene-1-carboxylic Acid {(S)-2-naphthylen-2-yl-1-[3-oxo-1-(pyridine-2-sulfonyl)-azepan-4-ylcarbamoyl)-ethyl]-amide

Following the procedures of Example 4e-h except substituting N-Boc-phenylalanine for N-(t-butoxycarbonyl)-3-(2-naphthyl)-L-alanine of Example 4e the title compound was prepared: MS 572 (M+H⁺).

### Example 7

### Preparation of Naphthylene-1-carboxylic Acid {(S)-2-naphthylen-2-yl-1-[3-oxo-1-(pyridine-2-sulfonyl)-azepan-4-ylcarbamoyl)-ethyl]-amide

Following the procedures of Example 4e-h except substituting N-Boc-phenylalanine for N-(t-butoxycarbonyl)-3-(2-naphthyl)-L-alanine of Example 4e and naphthoic acid for quinoline-8-carboxylic acid of Example 4g the title compound was prepared: MS 571 (M+H⁺).

### Example 8

### Preparation of Quinoline-2-carboxylic Acid {(S)-1-[3-oxo-1-(pyridine-2-sulfonyl)-azepan-4-ylcarbamoyl]-2-phenyl-ethyl}-amide

Following the procedures of Example 4e-h except substituting N-Boc-phenylalanine for N-(t-butoxycarbonyl)-3-(2-naphthyl)-L-alanine of Example 4e and quinoline-2-carboxylic acid for quinoline-8-carboxylic acid of Example 4g the title compound was prepared. Purification of the diastereomers by HPLC provided the two diastereomers of the title compound as solids (first: 40 mg: second: 43 mg): MS(ESI) 537.8 (M+H)⁺.

### Example 9

### Preparation of Benzofuran-2-carboxylic Acid {(S)-1-[3-oxo-1-(pyridine-2-sulfonyl)-azepan-4-ylcarbamoyl]-2-phenyl-ethyl}-amide

a.) {(S)-1-[3-hydroxy-1-(pyridine-2-sulfonyl)-azepan-4-ylcarbamoyl]-2-phenyl-ethyl}-carbamic Acid Tert-butyl Ester

To a solution of the compound of Example 4d (19 mg, 0.070 mmol) in CH₂Cl₂ was added N-Boc-phenylalanine (27.9 mg, 0.106 mmol), 1-hydroxybenzotriazole (16.1 mg, 0.12 mmol), and P-EDC (140 mg, 0.14 mmol) in CH₂Cl₂ . After shaking at room temperature overnight, the mixture was treated with PS-Trisamine. After shaking for another 2 hours, the mixture was filtered and concentrated to yield the title compound as a solid. MS (ESI) 518.87 (M+H)⁺.

b.) (S)-2-Amino-N-[3-hydroxy-1-(pyridine-2-sulfonyl)-azepan-4-yl]-3-phenyl-proprionamide

To a stirring solution of the compound of Example 9a (34 mg, 0.07 mmol) in CH₂Cl₂ (0.50 ml) was added HCl (4M in dioxane) (0.165 ml). After stirring at room temperature for 30 minutes, the mixture was concentrated, giving a white solid. The white solid was azeotroped with toluene then treated with MP-carbonate (0.35 mmol) in methanol. After four hours of shaking, the mixture was filtered and concentrated to give the title compound as a solid. MS(ESI) 418.91 (M+H)⁺.

c.) Benzofuran-2-carboxylic Acid {(S)-1-[3-hydroxy-1-(pyridine-2-sulfonyl)-azepan-4-ylcarbamoyl]-2-phenyl-ethyl}-amide

To a solution of the compound of Example 9b (27 mg, 0.070 mmol) in CH₂Cl₂ was added benzofuran-2-carboxylic acid (17.0 mg, 0.106 mmol), 1-hydroxybenzotriazole (16.1 mg, 0.12 mmol), and P-EDC (140 mg, 0.14 mmol ) in CH₂Cl₂ . After shaking at room temperature overnight, the mixture was treated with PS-Trisamine. After shaking for another 2 hours, the mixture was filtered and concentrated to yield the title compound as a solid. MS (ESI) 562.73 (M+H)⁺.

d.) Benzofuran-2-carboxylic Acid {(S)-1-[3-oxo-1-(pyridine-2-sulfonyl)-azepan-4-ylcarbamoyl]-2-phenyl-ethyl}-amide

To a stirring solution of the compound of Example 9c (37 mg, 0.07 mmol) in CH₂Cl₂ (0.5 ml) was added Dess-Martin reagent (45 mg, 0.105 mmol). After stirring for 30 minutes, solutions of sodium thiosulfate (10% in water, 0.50 ml) and saturated aqueous sodium bicarbonate (0.50 ml) were added simultaneously to the reaction. The mixture was then extracted with dichloromethane (2 times). The organic layer was dried, filtered, and concentrated. The residue was purified by HPLC to yield the two diastereomers of the title compound as solids (first eluting: 7 mg, second eluting: 5.5 mg). MS (ESI) 560.81 (M+H)⁺.

### Example 10

### Preparation of Benzofuran-2-carboxylic Acid {(S)-2-naphthylen-2-yl-1-[3-oxo-1-(pyridine-2-sulfonyl)-azepan-4-ylcarbamoyl)-ethyl]-amide

Following the procedures of Examples 9a-d except substituting N-(t-butoxycarbonyl)-3-(2-naphthyl)-L-alanine for N-Boc-phenylalanine the title compound was purified to yield two diastereomers as solids (first eluting: 5.3 mg, second eluting: 3.3 mg): MS(ESI): 610.8 (M+H)⁺.

### Example 11

### Preparation of Benzofuran-2-carboxylic Acid {(S)-2-benzyloxy-1-[3-oxo-1-(pyridine-2-sulfonyl)-azepane-4-ylcarbamoyl]-ethyl}-amide

Following the procedure of Example 9a-d, except substituting N-Boc-O-benzyl-L-serine in step 9a the title compound was prepared as a mixture of diastereomers. To a solution of benzofuran-2-carboxylic acid {(S)-2-benzyloxy-1-[3-oxo-1-(pyridine-2-sulfonyl)-azepane-4-ylcarbamoyl]-ethyl}-amide (90 mg) in ethyl acetate (2 mL) was added 10% Pd/C (50 mg). Upon hydrogenolysis of approximately 50% of the starting benzyl ether the reaction was filtered and concentrated. Purification of this 4 component mixture by HPLC provided the first eluting diastereomer of the title compound (1 mg) and the second eluting diastereomer of the title compound (0.3 mg): MS(ESI): 590.94(M+H)⁺.

### Example 12

### Preparation of Benzofuran-2-carboxylic Acid {(S)-3-methyl-1-[(2,2′,4-trideuterio)-3-oxo-1-(pyridine-2-sulfonyl)-azepan-4-ylcarbamoyl]-butyl}amide

a.) Benzofuran-2-carboxylic Acid {(S)-3-methyl-1-[3-hydroxy-1-(pyridine-2-sulfonyl)-azepan-4-ylcarbamoyl]-butyl}amide

To a solution of (S)-2-amino-4-methyl-pentanoic acid [3-hydroxy-1-(pyridine-2-sulfonyl)-azepan-4-yl]-amide of Example 1i (0.15 g) in dichloromethane was added TEA (0.11 mL), HOBt (49 mg), EDC (69 mg) and benzofuran-2-carboxylic acid (58 mg). The reaction was stirred until complete. Workup and column chromatography (5% methanol:ethyl acetate) provided the title compound: MS(EI) 529 (M+H⁺).

b.) Benzofuran-2-carboxylic Acid {(S)-3-methyl-1-[3-oxo-1-(pyridine-2-sulfonyl)-azepan-4-ylcarbamoyl]-butyl}amide

To a solution of the alcohol of Example 11a (0.11 g) in DMSO was added TEA (0.17 mL) and pyridine sulfur trioxide complex (99 mg). The reaction was stirred at room temperature for approximately 2 hours whereupon it was partitioned between ethyl acetate and water. The organic layer was washed with brine, dried, filtered and concentrated. Column chromatography of the residue (10% CH₃OH:EtOAc) provided 75 mg of the title compound as a mixture of diastereomers: ¹H NMR (CDCl₃): δ 1.0 (m, 6H), 1.5-2.1 (m, 5H), 2.2 (m, 2H), 2.7 (m, 1H), 3.7 (dd, 1H), 4.0 (m, 1H), 4.7 (m, 2H), 5.0 (m, 1H), 7.2-7.3 (m, 3H), 7.4 (m, 4H), 7.6 (m, 1H), 8.0 (m, 2H), 8.7 (m, 1H); MS(EI): 527 (M+H⁺, 40%).

c.) Benzofuran-2-carboxylic Acid {(S)-3-methyl-1-[(2,2′,4-trideuterio)-3-oxo-1-(pyridine-2-sulfonyl)-azepan-4-ylcarbamoyl]-butyl}amide

To a solution of benzofuran-2-carboxylic acid {(S)-3-methyl-1-[3-oxo-1-(pyridine-2-sulfonyl)-azepan-4-ylcarbamoyl]-butyl}amide of Example 11b (0.03 g) in D₂O:CD₃OD (0.4:4 mL) was added triethylamine (0.04 mL). The reaction was heated to reflux for 2 hours whereupon it was concentrated and dried under vacuum. The residue was the redissolved in the same mixture and heated to reflux overnight. The reaction was concentrated and the residue purified by column chromatography (5% methanol:dichloromethane) to provide the title compound (0.02 g): ¹HNMR: δ 1.0 (m, 6H), 1.5-2.2 (m, 6H), 2.7 (m, 1H), 4.1 (m, 1H), 4.7 (m, 2H), 7.4-8.0 (m, 8H), 8.7 (m, 1H); MS(EI): 529 (M⁺, 45%).

The diastereomeric mixture was separated by HPLC to provide the faster eluting diastereoemer: MS(EI): 530 (M+H⁺, 100%) and the slower eluting diastereomer: MS(ET): 530 (M+H⁺, 100%).

The above specification and Examples filly disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A method of inhibiting cathepsin L, comprising administering to a patient in need thereof an effective amount of a compound of Formula I: 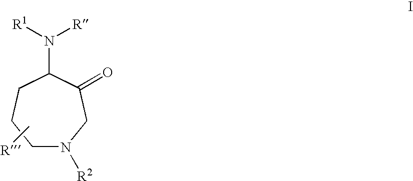
wherein:
R¹ is selected from the group consisting of: 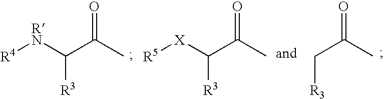
R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 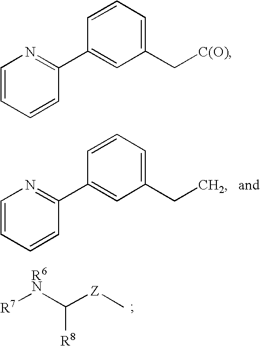
R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;
R³ and R′ may be connected to form a pyrrolidine, piperidine or morpholine ring;
R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;
R⁶ is selected from the group consisting of: H, C₁₋₆alkyl, C₃₋₆cycloalkyl-C₀₋₆alkyl, Ar—C₀₋₆alkyl, or Het-C₀₋₆alkyl;
R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, and R¹⁰R¹⁴NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloalkyl-C₀₋₆alkyl, Ar—C₀₋₆alkyl and Het-C₀₋₆alkyl;
R¹⁰ is selected from the group consisting of: C₁₋₆alkyl, C₃₋₆cycloalkyl-C₀₋₆alkyl, Ar—C₀₋₆alkyl and Het-C₀₋₆alkyl;
R¹¹ is selected from the group consisting of: H, C₁₋₆alkyl, Ar—C₀₋₆alkyl, and Het-C₀₋₆alkyl;
R¹² is selected from the group consisting of: H, C₁₋₆alkyl, Ar—C₀₋₆alkyl, and Het-C₀₋₆alkyl;
R¹³ is selected from the group consisting of: H, C₁₋₆alkyl, Ar—C₀₋₆alkyl, and Het-C₀₋₆alkyl;
R¹⁴ is selected from the group consisting of: H, C₁₋₆alkyl, Ar—C₀₋₆alkyl, and Het-C₀₋₆alkyl;
R′ is selected from the group consisting of: H, C₁₋₆alkyl, Ar—C₀₋₆alkyl, and Het-C₀₋₆alkyl;
R″ is selected from the group consisting of: H, C₁₋₆alkyl, Ar—C₀₋₆alkyl, or Het-C₀₋₆alkyl;
R′″ is selected from the group consisting of: H, C₁₋₆alkyl, C₃₋₆cycloalkyl-C₀₋₆alkyl, Ar—C₀₋₆alkyl, and Het-C₀₋₆alkyl;
X is selected from the group consisting of: CH₂, S, and O; and
Z is selected from the group consisting of: C(O) and CH₂;
and pharmaceutically acceptable salts, hydrates and solvates thereof.

2. A method according to claim 1 wherein in said compound R¹ is

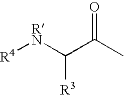

3. A method according to claim 2 wherein in said compound R³ is selected from the group consisting of: C₁₋₆alkyl and Ar—C₀₋₆alkyl.

4. A method according to claim 3 wherein in said compound R³ is selected from the group consisting of: isobutyl, napthalen-2-ylmethyl, benzyl, and benzyloxymethyl.

5. A method according to claim 2 wherein in said compound R⁴ is R⁵C(O)—.

6. A method according to claim 5 wherein in said compound R⁵ is selected from the group consisting of: C₁₋₆alkyl, Ar—C₀₋₆alkyl and Het-C₀₋₆alkyl.

7. A method according to claim 6 wherein in said compound R⁵ is selected from the group consisting of: quinolinyl, isoquinolinyl, and benzofuranyl.

8. A method according to claim 6 wherein in said compound R⁵ is selected from the group consisting of: quinolin-2-yl, quinolin-4-yl, quinolin-8-yl, isoquinolin-1-yl, napthalen-1-yl, and benzofuran-2-yl.

9. A method according to claim 1 wherein in said compound R′ is H.

10. A method according to claim 1 wherein in said compound R″ is H.

11. A method according to claim 1 wherein in said compound R′″ is H.

12. A method according to claim 1 wherein in said compound R″ and R′″ are both H.

13. A method according to claim 1 wherein in said compound R² is R⁹SO₂.

14. A method according to claim 13 wherein in said compound R⁹ is Het-C₀₋₆alkyl.

15. A method according to claim 14 wherein in said compound R⁹ is selected from the group consisting of: pyridinyl and 1-oxy-pyridinyl.

16. A method according to claim 15 wherein in said compound R⁹ is selected from the group consisting of: pyridin-2-yl and 1-oxy-pyridin-2-yl

17. A method according to claim 1 wherein in said compound:
R¹ is 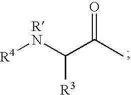
R² is R⁹SO₂;
R³ is selected from the group consisting of: isobutyl, napthalen-2-ylmethyl, benzyl, and benzyloxymethyl;
R⁴ is R⁵C(O);
R⁵ is selected from the group consisting of: quinolin-2-yl, quinolin-4-yl, quinolin-8-yl, isoquinolin-1-yl, napthalen-1-yl, and benzofuran-2-yl.
R⁹ is selected from the group consisting of: pyridin-2-yl and 1-oxy-pyridin-2-yl;
R′ is H
R″ is H; and
R′″ is H;

18. A method according to claim 17 wherein said compound is selected from the group consisting of:
Quinoline-8-carboxylic acid {(S)-3-methyl-1-[3-oxo-1-(pyridine-2-sulfonyl)-azepan-4-ylcarbamoyl]-butyl}amide;
Quinoline-4-carboxylic acid {(S)-3-methyl-1-[3-oxo-1-(pyridine-2-sulfonyl)-azepan-4-ylcarbamoyl]-butyl}amide;
Isoquinoline-1-carboxylic acid {(S)-3-methyl-1- [3-oxo-1-(pyridine-2-sulfonyl)-azepan-4-ylcarbamoyl]-butyl}amide;
Quinoline-8-carboxylic acid {(S)-2-naphthylen-2-yl-1-[3-oxo-1-(pyridine-2-sulfonyl)-azepan-4-ylcarbamoyl)-ethyl]-amide;
Naphthylene-1-carboxylic acid {(S)-2-naphthylen-2-yl- 1-[3-oxo-1-(pyridine-2-sulfonyl)-azepan-4-ylcarbamoyl)-ethyl]-amide;
Quinoline-8-carboxylic acid {(S)-1-[3-oxo-1-(pyridine-2-sulfonyl)-azepan-4-ylcarbamoyl]-2-phenyl-ethyl}-amide;
Naphthylene-1-carboxylic acid {(S)-1-[3-oxo-1-(pyridine-2-sulfonyl)-azepan-4-ylcarbamoyl]-2-phenyl-ethyl}-amide;
Quinoline-2-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-2-naphthylen-2-yl-1-[3-oxo-1-(pyridine-2-sulfonyl)-azepan-4-ylcarbamoyl)-ethyl]-amide; and
Benzofuran-2-carboxylic acid {(S)-2-benzyloxy-1-[3-oxo-1-(pyridine-2-sulfonyl)-azepane-4-ylcarbamoyl]-ethyl}-amide.

19. A method of treating a disease characterized by cancer metastasis comprising inhibiting said cancer metastasis by administering to a patient in need thereof an effective amount of a compound according to claims 1 to 18.

20. A method of treating a disease characterized by positive selection of CD4⁺T⁻ cells by cortical thymic epithelial cells comprising inhibiting said positive selection of CD4⁺T⁻ cells by cortical thymic epithelial cells by administering to a patient in need thereof an effective amount of a compound according to claims 1 to 18.

21. A method of treating a disease characterized by tissue destruction by a macrophage, comprising inhibiting said tissue destruction by administering to a patient in need thereof an effective amount of a compound according to claims 1 to 18.

22. A method of treatment according to claim 21 wherein said macrophage is a lung macrophage.

23. A method of treatment according to claim 21 wherein said disease is selected from the group consisting of: asthma, chronic obstructive pulmonary disease (COPD), and emphysema.

24. Use of a compound according to any one of claims 1 to 18 in the manufacture of a medicament for use in inhibiting cathepsin L.

25. Use of a compound according to any one of claims 1 to 18 in the manufacture of a medicament for use in treating a disease characterized by cancer metastasis.

26. Use of a compound according to any one of claims 1 to 18 in the manufacture of a medicament for use in treating a disease characterized by positive selection of CD4⁺T⁻ cells by cortical thymic epithelial cells.

27. Use of a compound according to any one of claims 1 to 18 in the manufacture of a medicament for use in treating a disease characterized by tissue destruction by a macrophage.

28. A use according to claim 27 wherein said macrophage is a lung macrophage.

29. A use according to claim 28 wherein said disease is selected from the group consisting of: asthma, chronic obstructive pulmonary disease (COPD), and emphysema.
